(19) United States
(12) Patent Application Publication
(10) Pub. No.: US 2026/0026819 A1
(43) Pub. Date: Jan. 29, 2026

(54) DEVICE AND METHOD FOR ANEURYSM OCCLUSION UTILIZING A STAND-ALONE NECK FLOW DIVERTER

(71) Applicant: Carlos Jimenez, Medellin (CO)

(72) Inventor: Carlos Jimenez, Medellin (CO)

(21) Appl. No.: 18/786,784

(22) Filed: Jul. 29, 2024

(51) Int. Cl. A61B 17/12; A61B 17/00; A61B 90/00

(52) U.S. Cl. CPC A61B 17/12113; A61B 17/12145; A61B 2017/00867; A61B 2090/3966

(57) ABSTRACT

A flow-diverting device is designed for endovascular aneurysm repair by occluding lesions without high metal load or aneurysm wall manipulation. It features a spiral barrier that covers the aneurysm neck and diverts blood flow to the aneurysm sac, promoting thrombosis. The freestanding, endovascular implant includes stent-like crowns for stable fixation and precise positioning. Made from a shape memory alloy, the device is constrained in a linear form within a delivery catheter and reverts to its functional shape upon catheter retraction.

### FIELD OF THE INVENTION

The presented invention falls within the domain of implantable medical devices, specifically addressing the closure of focal defects in arterial walls, predominantly saccular brain aneurysms. This device and method are applicable via minimally invasive techniques within the scope of endovascular interventions. Through this invention, an operator can implant a barrier formed from a filament that takes the shape of an infinite spiral, achieving precise positioning of the barrier to treat focal vascular wall defects, such as aneurysms. More precisely, this invention functions as a flow diverter device specifically targeting the neck of the lesion and is designed to treat a wide range of aneurysms effectively.

The device is a barrier that is organized in the shape of an infinite spiral, integrated into a stent-like platform, designed to deploy its flow-diverting effect precisely and exclusively in the neck area of the aneurysm, without impacting the surrounding circulation, during an endovascular procedure. In short, this invention consists of a filament-based stent-like platform with a spiral barrier, and comprises the following components:

Filament-based stent-like platform: A single filament, initially housed within a carrier catheter. Upon deployment, the filament assumes the shape of a stent-like platform. This platform serves as a structural support within the vascular system.

Spiral barrier: Continuously connected to the stent-like platform, in some point the filament takes the form of an infinite spiral. Functions as a barrier to control blood flow through the neck of the lesion.

This invention combines a flexible filament-based stent with an intricately designed spiral barrier, offering potential applications in vascular interventions.

### DESCRIPTION OF THE PRIOR ART

### Defects in the Vascular Wall: Basic Concepts

Blood vessels are tubular organs arranged in an interconnected network, designed to transport blood to various tissues in the human body. Defects in the vascular wall, such as aneurysms, are lesions that can occur anywhere in the circulatory system but are most frequently found in the aorta and cerebral circulation. These defects result in the deformation of the normal cylindrical wall of an artery, creating a blind sac-like structure that is structurally weaker than the parent vessel and can eventually rupture, leading to life-threatening hemorrhage.

The initial histopathological event in aneurysm formation is the disruption of the internal elastic membrane due to hemodynamic forces, typically at arterial bifurcation sites where hemodynamic stress is high. Over time, the layers of the arterial wall undergo degenerative changes, resulting in a weakened aneurysm wall that lacks smooth muscle cells in certain areas of the middle layer, which is crucial for the structural integrity of an artery. This structural weakness is what potentially leads to the rupture of aneurysms.

The neck of the aneurysm is the area of the vascular wall where histological changes begin due to hemodynamic forces, and the saccular lesion forms. Once formed, blood flow enters the aneurysm cavity through the neck, impacting its wall and often leading to rupture. Therefore, any intervention aimed at preventing aneurysm rupture must focus on imposing a mechanical barrier at the neck, either by blocking the blood flow from contacting the lesion wall or by restricting its passage through the neck. The absence or extreme restriction of flow through the neck, below a threshold of approximately 40% or more, protects the aneurysm wall from harmful mechanical forces, leading to thrombosis and closure of the lesion, thus eliminating the risk of rupture.

For more detailed information, refer to the study by Pereira V M, Brina O, Gonzalez A M, Narata A P, Ouared R, Karl-Olof L. titled “Biology and hemodynamics of aneurysmal vasculopathies,” published in the European Journal of Radiology in October 2013.

### Intravascular Stents Without a Covered Segment

These devices are designed to create a mechanical barrier that partially blocks the migration of solid or liquid elements from the defective wall of an artery, particularly from the lesion being treated. In the context of focal arterial wall lesions, such as aneurysms, these stents are typically used as adjuncts rather than primary treatments. Their advantages include low metal content, high porosity, excellent navigability, and good adaptability and tolerance within arterial vessels.

Studies have highlighted the importance and application of these stents:
Nakasaki et al. (2017) discussed that the diameter of the aneurysm neck is an independent predictor of progressive occlusion when using stent-assisted coiling, indicating the critical role of stents in enhancing treatment efficacy for aneurysms.
Lubicz et al. (2017) examined the long-term outcomes of stent-assisted coiling in wide-neck bifurcation aneurysms. Their findings supported the use of stents in achieving better occlusion and stability in these complex cases.

Covered stents are designed to address focal defects in arterial walls by obstructing blood flow contact with the defect. However, positioning a covered segment precisely over the focal defect can be challenging. The non-porous covered area may inadvertently occlude vessel segments from which branches originate, potentially causing ischemic complications.

Partially Covered Stents: In stents with only a covered segment, achieving precise positioning of the covered area over the lesion is particularly difficult. This requires the operator to manipulate the stent to rotate it on its axis, aiming for accurate alignment of the covered segment in the coronal, axial, sagittal, and azimuthal planes. Such manipulation is known to cause severe endothelial damage and provoke a spastic reaction in the arterial wall. Therefore, while partially covered stents offer some benefits, their application is limited by the technical challenges associated with their precise deployment.

Flow Diversion Devices: These devices are characterized by low porosity and are designed to redirect blood flow away from the focal defect being treated. While they effectively prevent blood from contacting the defect, they have several drawbacks. The high number of wires required to create the device's porous structure increases the overall metal mass, which can lead to intimal hyperplasia. Additionally, this increased metal mass may result in the occlusion of adjacent vessels, potentially causing ischemic complications.

Intrasaccular Flow Diversion Devices: These devices are implanted endovascularly within the aneurysmal sac and consist of a porous mesh that occupies space within the lesion. They induce thrombosis by partially restricting blood flow into the aneurysm, promoting clot formation and ultimately aiding in the exclusion of the aneurysm from the circulatory system. Some challenges are associated with these devices; one significant issue is that manipulation of the device within the aneurysmal cavity can increase the risk of rupture. Additionally, there have been reports of device migration from its original position, where it may function as a foreign body that can be displaced by blood flow, potentially generating fatal complications and compromising the treatment outcome.

Comparison of the Present Invention with the Previous State of the Art:

There is a clear need for a more effective and safer treatment method for focal defects in the arterial wall, such as aneurysms. The proposed solution aims to seal the neck of the lesion while preserving the integrity of the surrounding vessel, thereby avoiding contact with the delicate walls of the aneurysmal cavity, which poses a significant risk of iatrogenic rupture. This approach facilitates tissue regeneration through reendothelialization, leading to permanent repair, and minimizes the risk of recanalization, which could result in persistent aneurysm.

Covered stents, while commonly used, present several challenges. Their rigidity can complicate navigation during deployment, and they may occlude adjacent vascular branches. Furthermore, these devices can induce progressive intimal hyperplasia, potentially leading to thrombosis of the arterial lumen (Romaguera R, Waksman R. Covered stents for coronary perforations: is there enough evidence? Catheter Cardiovasc Interv. 2011 Aug. 1; 78 (2): 246-53. doi: 10.1002/ccd.23017. Epub 2011 Jul. 15. PMID: 21766425).

Endoluminal flow diverter devices are often associated with intimal hyperplasia due to their high metal content, typically around 30%. Additionally, these devices can lead to thrombosis in the vessels adjacent to the focal defect being treated (Sindeev S, Prothmann S, Frolov S, Zimmer C, Liepsch D, Berg P, Kirschke J S, Friedrich B. Intimal Hyperplasia After Aneurysm Treatment by Flow Diversion. World Neurosurg. 2019 February; 122: e577-e583).

Intrasaccular Flow Diverter Devices: These devices involve the implantation of a foreign body within the aneurysmal sac, which presents a risk of contact with the delicate walls of the lesion, potentially leading to rupture. Additionally, the device may become displaced due to residual blood flow entering the aneurysm, a phenomenon known as the “inverted umbrella effect.” Moreover, these devices have limited applicability for non-bifurcation aneurysms (Akhunbay-Fudge C Y, Deniz K, Tyagi A K, Patankar T. Endovascular treatment of wide-necked intracranial aneurysms using the novel Contour Neurovascular System: a single-center safety and feasibility study. J Neurointerv Surg. 2020 October; 12 (10): 987-992. doi: 10.1136/neurintsurg-2019-015628. Epub 2020 Jan. 22. PMID: 31974281; PMCID: PMC7509519).

Furthermore, several reports indicate that these devices can be dislodged by blood flow (Bhogal P, Udani S, Cognard C, Piotin M, Brouwer P, Sourour N A, Andersson T, Makalanda L, Wong K, Fiorella D, Arthur A S, Yeo L L, Soderman M, Henkes H, Pierot L. Endosaccular flow disruption: where are we now? J Neurointerv Surg. 2019 October; 11 (10): 1024-1025. doi: 10.1136/neurintsurg-2018-014623. Epub 2019 Jun. 13. PMID: 31197026).
The following concepts highlight the advantages of the method and the device described in the present invention in comparison with the previously used methods and devices:
The method and device described in this invention offer several advantages over previously used methods and devices. Specifically, it allows the operating physician to treat focal defects in an artery wall, such as aneurysms. By implanting a barrier designed in the shape of an infinite spiral, that restricts flow exclusively in the neck area of the lesion, the method avoids affecting the rest of the artery. Importantly, taking into account that the device does not enter the interior of the aneurysm sac at any time, this approach prevents intraoperative rupture of the vessel and the lesion, as well as occlusion and thrombosis of the parent artery and adjacent branches. The technique achieves flow diversion specifically at the lesion focus (e.g., the neck of an aneurysm) without involving non-diseased areas of the arterial wall o sacrificing adjacent arterial branches.

The invention offers several advantages over the method of using a single-layer stent with uniform porosity in the parent vessel for treating focal defects. Specifically:

Metal-to-artery ratio: Unlike the single-layer stent, this method does not impose a high metal-to-artery ratio, given that its entire structure consists of a single filament that can adopt various shapes, reducing the risk of reactive intimal hyperplasia, stenosis, or thrombosis in the vessel where the stent is implanted.

Preservation of adjacent vessels: This approach eliminates the risk of occlusion at the ostium or origin of vessels adjacent to the neck of the aneurysm.

The potential advantages of this invention, in comparison to deploying a stent in the parent vessel with pre-shaped areas of different porosity, are as follows:

Metal-to-artery ratio: Unlike the stent method, our approach avoids imposing a high metal-to-artery ratio on the vascular endothelium. This reduces the risk of intimal hyperplasia, stenosis, or thrombosis in the vessel where the stent is deployed.

Preservation of adjacent vessels: Our invention eliminates the risk of occlusion at the ostium or origin of vessels adjacent to the focal defect in the vascular wall (e.g., an aneurysm).

Advantages of this invention compared to deploying a stent in the parent vessel with pre-shaped areas of different porosity: Again, metal-to-artery ratio: Unlike the stent method, this approach avoids imposing a high metal-to-artery ratio on the vascular endothelium. This reduction in metal content minimizes the risk of complications such as intimal hyperplasia, stenosis, or thrombosis in the vessel where the stent is deployed. Regarding the preservation of adjacent vessels: this invention eliminates the risk of occlusion at the ostium or origin of vessels adjacent to the focal defect in the vascular wall (e.g., an aneurysm). By implanting a barrier with a flow diversion effect exclusively in the neck of the aneurysm and from the lumen of the parent vessel, the operator maintains the integrity of the parent vessel walls and its branches.

Advantages of this invention compared to deploying a stent in the parent vessel with areas of different porosity created by modifying the stent surface after its expansion: once again, in relation to metal-to-artery ratio, unlike the stent method, this approach avoids imposing a high metal-to-artery ratio on the vascular endothelium. By doing so, the operator reduces the risk of complications such as intimal hyperplasia, stenosis, or even thrombosis in the vessel where the stent is deployed. On the other hand, regarding the preservation of adjacent vessels, this invention eliminates the risk of occlusion at the ostium or origin of vessels adjacent to the focal defect in the vascular wall (e.g., an aneurysm). By implanting a barrier with a flow diversion effect exclusively in the neck of the aneurysm and from the lumen of the parent vessel, the operator maintains the integrity of the parent vessel walls and its branches.

Compared to the conventional method of deploying a stent with a radial expansion element and an accompanying lateral expanding occluding element, this invention offers several key advantages:

Simplified Procedure: Instead of implanting an occluding element directly into the focal defect of the vascular wall (as required in aneurysms), our device streamlines the process. It involves a single moment of implantation, exclusively targeting flow diversion at the aneurysm neck.

Risk Reduction: By avoiding direct contact with the focal defect walls and eliminating the need for internal manipulation, it's significantly reduced the risk of iatrogenic rupture of the arterial lesion. All procedures occur externally, minimizing the danger associated with intraoperative complications.

This approach focuses on efficient flow diversion without entering the aneurysm, enhancing safety and effectiveness. Moreover, it is crucial to highlight that the manipulation of the vascular endothelial tissue and the associated metal load remain minimal. As a result, the risk of endothelial hyperplasia and thromboembolic complications is significantly reduced.

Advantages of this invention over intra-saccular occlusion devices for aneurysms: compared to the conventional method of deploying an occluding device directly inside the aneurysm sac, this approach offers significant advantages:

Non-invasive aneurysm sac procedure: This method eliminates the need for direct interaction with the sac itself. There is no contact between the device and the sac walls, reducing the risk of iatrogenic rupture of the lesion.

Avoidance of intrasaccular foreign bodies: Unlike traditional approaches, this invention does not leave a foreign body inside the aneurysm. This minimizes the risk of blood flow displacing or dislodging the device from its intended location.

The primary purpose of this device is to redirect blood flow specifically within the aneurysm neck. By doing so, it prevents excessive blood flow into the aneurysm sac, reducing the risk of rupture. The device achieves flow diversion without compromising the integrity of the parent vessel (the artery from which the aneurysm arises). This preservation is crucial to maintain overall blood supply and prevent damage to the vessel. Unlike some other treatments, this approach avoids implanting foreign bodies directly into the aneurysm sac. By doing so, it minimizes the risk of complications associated with foreign material within the aneurysm. The benefits extend to reducing several risks:
Parent vessel damage: by respecting the parent vessel, the risk of vessel injury during the procedure is minimized.
Arterial occlusion: the device avoids blocking other arteries related to the aneurysm, ensuring continued blood flow.
Lesion wall manipulation: manipulating the aneurysm wall can lead to complications; this approach eliminates such manipulation.
Intraoperative rupture prevention: the most dreaded complication during aneurysm intervention is intraoperative rupture of the lesion.

This device strikes a delicate balance between effective flow diversion and minimizing risks. Its design and application aim to achieve successful outcomes while prioritizing patient safety.

### BRIEF DESCRIPTION OF THE INVENTION

Purpose of the Invention. The objective of the present invention is to provide a method and device for occluding focal defects in the arterial wall, such as aneurysms. This invention encompasses a method for loading, navigating, and deploying a low-porosity barrier integrated with a conventional high-porosity stent-like component, which serves as a support platform for implantation in the affected artery. This approach is particularly useful for treating both sidewall and bifurcation aneurysms.

Device Functionality. The device operates as a flow diverter barrier, integrated with a high-porosity stent-like element, and is navigable and implantable via endovascular methods. The device can be fully retrievable before release, according to the operator's intent. It can be generally described as an occluding device implanted in an artery during an interventional endovascular procedure.

Radiopaque Features: Radiopaque labels or compounds may be incorporated into specific parts or structures of the device to facilitate location, positioning, and monitoring both during and after deployment in circulation.

Device Navigation and Deployment: The device consists of a single filament that adopts different predetermined shapes. During navigation, the device is loaded into a catheter in a straightened form. As the catheter is retracted, the filament assumes its designed shapes sequentially. This transformation occurs as the filament exits the catheter, allowing it to conform to the specific geometries in which it has been manufactured. The following descriptions provide exemplary, but not limiting, embodiments of the invention. Various functionalities can be achieved through combinations of parts, components, materials, and structures described herein.

Combinations and Additional Functionalities: A skilled operator or evaluator experienced in the endovascular field will understand how different components and structures can be combined to create additional devices and functionalities. This invention's versatility allows for numerous configurations to address various clinical needs.

The device features a flow-diverting barrier, in the form of an infinite spiral, specifically engineered to occupy the entire neck area of an aneurysm. This barrier is seamlessly integrated with a stent-like structure at both its distal and proximal ends. The entire structure is implanted within the parent vessel of the aneurysm, ensuring the barrier's stability and preventing its displacement by blood flow. It is particularly effective for treating both sidewall and bifurcation aneurysms.

### Device Composition.

Occluding spiral: A low-porosity barrier, formed from a filament in the shape of an infinite spiral, functions as a blood flow diverter, specifically designed to effectively cover the neck of an aneurysm.

Stent-Like Platform: A high-porosity, cylindrical support structure that stabilizes the occluding spiral, ensuring it remains in place against the aneurysm neck.

It is crucial to understand that both the infinite spiral forming the neck barrier and the stent-like structure stabilizing the device within the parent vessel of the aneurysm are derived from a single, continuous filament, ensuring uninterrupted structural integrity.

### Device Navigation and Deployment

The device is loaded into a catheter in in a straightened form and assumes its manufactured shape once the delivery catheter is retracted. The stent-like platform anchors against the vessel walls, preventing displacement by blood flow. Radiopaque markers are Incorporated to aid in precise positioning and monitoring during and after deployment. The device targets only the aneurysm neck, preserving normal blood flow in the parent vessel and preventing ischemic complications, and it is navigable and implantable using established endovascular techniques, making it practical for clinical use.

This invention provides a reliable and effective solution for treating sidewall aneurysms via a minimally invasive endovascular approach. The described embodiments offer flexibility in addressing various anatomical challenges, ensuring precise and stable occlusion of aneurysm necks while preserving the integrity of the surrounding vasculature.

### BRIEF DESCRIPTION OF THE DRAWINGS

FIG. 1 presents a top-down view of the entire device, highlighting its construction from a single filament extending from the distal to the proximal end. The figure also demonstrates the integration of four components connected seamlessly in a continuous structure.

FIG. 2 presents a lateral projection of the device, clearly delineating the four components arranged from distal to proximal: guide ring, distal crowns, spiral barrier and proximal crowns. The proximal end terminates in the detachment system, which is inserted into the tip of the delivery catheter.

FIG. 3 provides a detailed view of the distal and proximal ends, clearly demonstrating how the device is constructed from a single filament that continuously unfolds into various shapes, each serving distinct functions.

FIG. 4 offers a bottom-up view of the device, clearly illustrating the formation of the occlusive spiral barrier, which is essential for the device's functionality.

FIG. 5 again illustrates the device's construction from a single filament, highlighting its distal and proximal ends.

FIG. 6 provides a panoramic side projection of the device.

FIG. 7 demonstrates how the device transitions from a single filament into a tubular structure, making it perfectly adaptable to the geometry of a blood vessel.

FIG. 8, along with the subsequent figures, demonstrates the step-by-step deployment of the device, which consists of a filamentous structure navigating in a straight line within the delivery catheter. By retracting the catheter, the device is gradually released, and recovers its original conformation, typical of a filamentous shape memory alloy (SMA) structure undergoing a reversible shape memory effect (SME) in response to thermal stimuli. In this figure, only the guide ring has been deployed, enabling the operator to manipulate and align it with the center of the aneurysm neck.

FIG. 9 depicts the second deployed component, specifically the distal crowns, which form a tubular stent-like structure.

FIG. 10 presents the spiral barrier, the third component, which has been successfully released and now spans the entire width of the aneurysm neck.

FIG. 11 demonstrates the successful deployment of the fourth component, the proximal crowns with a stent-like design, via catheter retraction, revealing the device in its fully reconfigured state utilizing filament shape memory behavior, with its proximal end slightly extending beyond the catheter tip.

### DETAILED DESCRIPTION OF THE INVENTION

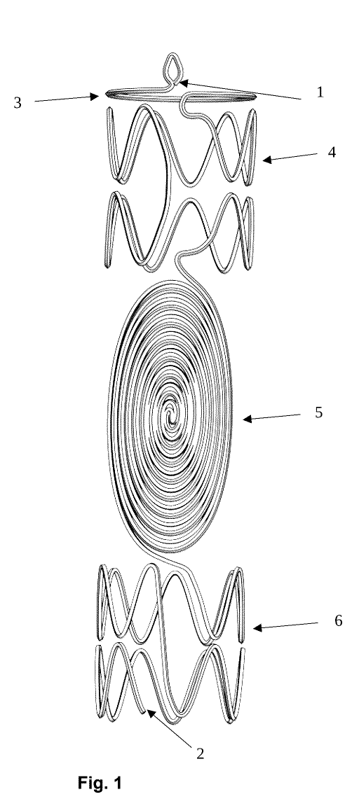

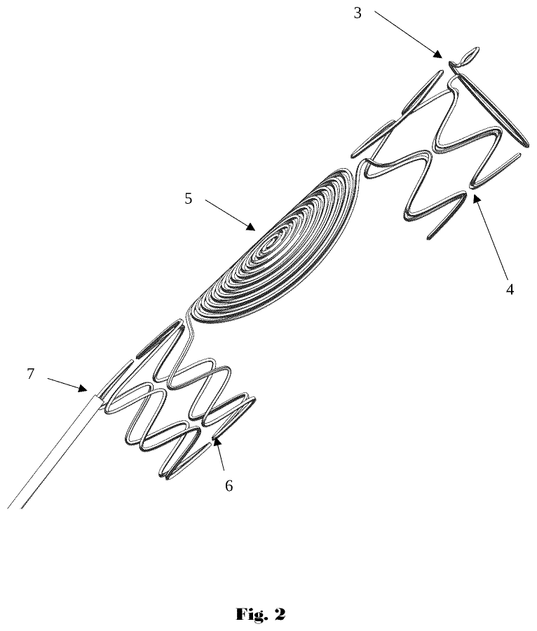

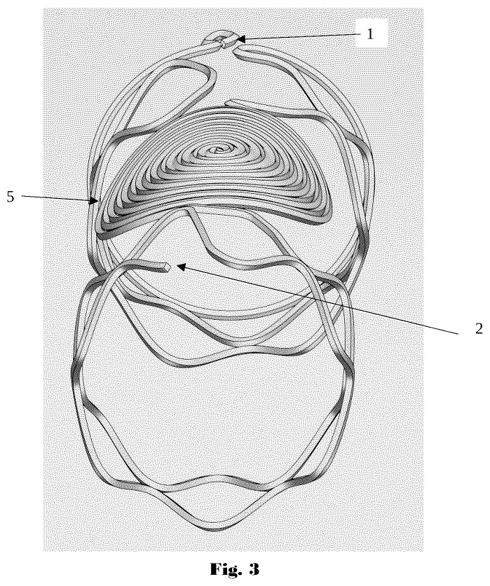

Taking the figures as a reference, the invention aims to correct vascular lesions, particularly aneurysms. It accomplishes this by occluding or isolating the lesion within the vascular wall. The device consists of one single filament that constitutes the entire device, allowing it to assume its final shape upon release from the catheter that contains and transports it straightened to its target site, when deployed by the specialist who performs the intervention, during an endovascular procedure, recovering its original conformation, typical of a filamentous shape memory alloy (SMA) structure undergoing a reversible shape memory effect (SME) in response to thermal stimuli. This single filament seamlessly and continuously forms the four components of the device, as illustrated in FIG. 1 and FIG. 2, listed below from distal to proximal, corresponding to the sequence in which the device is deployed from its delivery catheter: guide ring 3, distal stabilizing crowns 4, spiral barrier 5, proximal stabilizing crowns 6. The guide ring ensures the precise positioning of the spiral barrier, allowing it to span the entire length of the aneurysm neck. The spiral barrier is the component that effectively divert blood flow to the lesion. The two crowns prevents migration or detachment of the barrier. The device, comprising its four integrated components, ensures effective isolation of the aneurysm neck from blood flow. It is important to note that the filament constituting each of the four mentioned components can be fabricated from either a straight thread or a sinusoidal (wavy) thread, with the aim of enhancing its functionality.

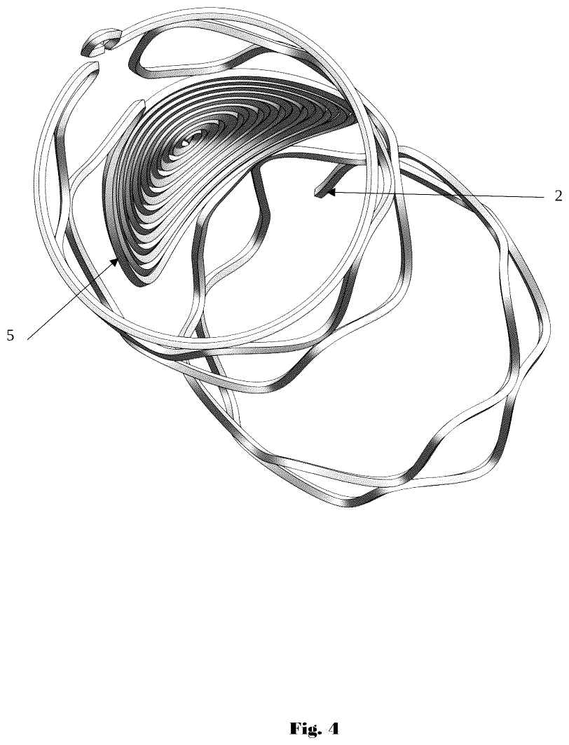

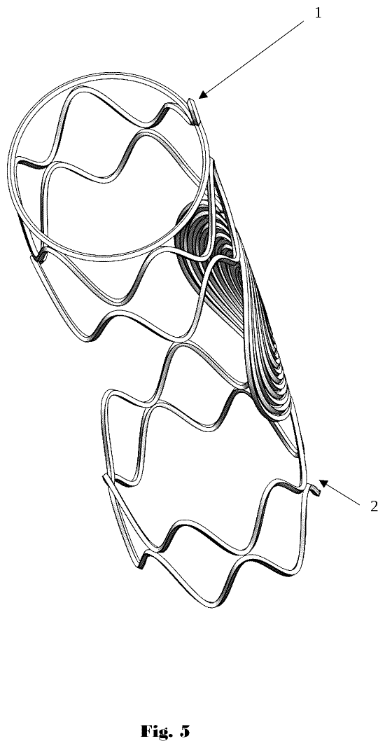

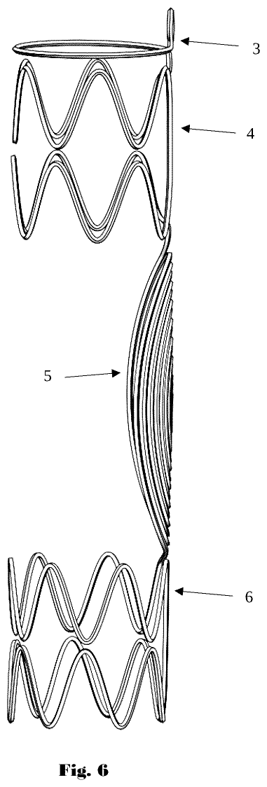

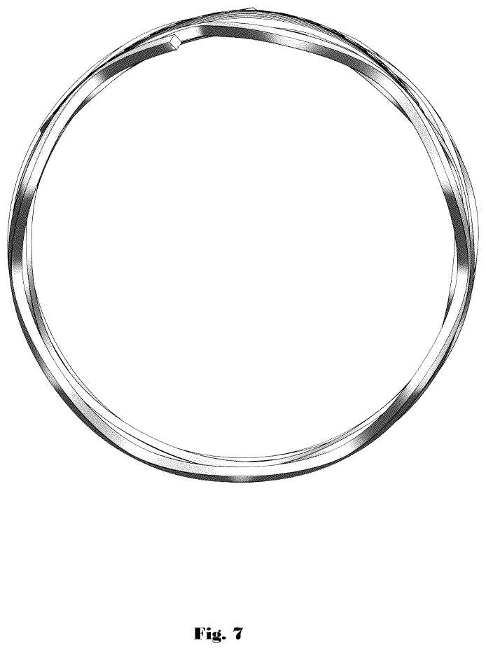

The four components are outlined in detail below:
The guide ring: It is a circular structure positioned distal to the aneurysm at a distance predefined by the operator. It features a discontinuity spanning 0.5 to 2 mm, resulting in two free ends. The first end 1 points distally, as illustrated in FIG. 1, FIG. 2, FIG. 3 and FIG. 5, and adopts a spherical shape 8, which during the manufacturing process is embedded with a highly radiopaque material, ensuring high visibility under X-ray imaging; this feature enables the operator to orient the device accurately, rotating the ring to align it with the center of the aneurysm neck, all under high-resolution fluoroscopy. The second end 2 is oriented towards the proximal part of the structure, aligned with the distal end, as shown in FIG. 1, FIG. 2, FIG. 3 and FIG. 5, creating a seamless connection between the ring and the distal crown. This design plays a critical role in maintaining the stability and functionality of the device.
Distal crowns: these are tubular stent-like structures, originating from the proximal end of the guide ring and originated from the same filament that makes it up. Their function is to ensure the stability of the device within the vascular lumen, when the filament is manufactured with a predefined cylindrical wavy shape, and it resembles the cells of a stent. This design ensures that it lies against the vascular walls with sufficient radial force, preventing the structure from being dislodged by the blood flow. The operator must predetermine the deployment location based on the patient's angiographic studies, ensuring that the distal structure closest to the aneurysm is positioned immediately distal to the neck of the lesion. Its proximal end is seamless continuous as a single filament with the spiral barrier, as illustrated in FIG. 1, FIG. 2, FIG. 3 and FIG. 5.
Spiral barrier: The structure 5 extends as a continuation of the same filament that conforms the distal stabilizing crowns 4, forming an infinite spiral with multiple loops, and seamlessly transitions into the proximal stabilizing crowns 3 at its proximal end. This spiral barrier is positioned by the operator to cover the neck of the aneurysm, utilizing the rotational and precise positioning capabilities of the guide ring. The filament's thickness determines the spiral's ability to divert blood flow adequately, leading to subsequent thrombosis of the aneurysm.
Proximal crowns: They are like the distal stabilizer crowns 6 and are formed as a continuous extension of the proximal end of the spiral barrier, as shown in FIG. 1. Their function is analogous to that of the distal structures: they act as stabilizing elements for the occluding spiral barrier.

By positioning the spiral barrier across the aneurysm neck area, it effectively diverts the inflow jet to the aneurysm. The spiral is a continuous component, seamlessly connected to both the distal and proximal stabilizing crowns as a single filament as shown in FIG. 1, FIG. 2 and FIG. 6. This ensures flexibility and structural integrity, as well as optimal and straightforward navigability and deployment, since, once the guide ring is properly positioned, aligned with the center of the neck of the aneurysm, the only thing the operator must do is gently release the single filament that makes up all and each of the components of the device, which acquire the shape that was previously given to them in the manufacturing process.

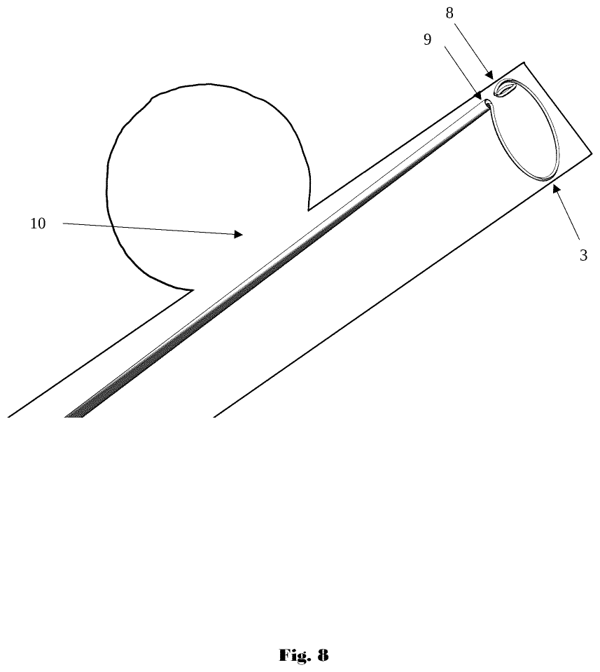

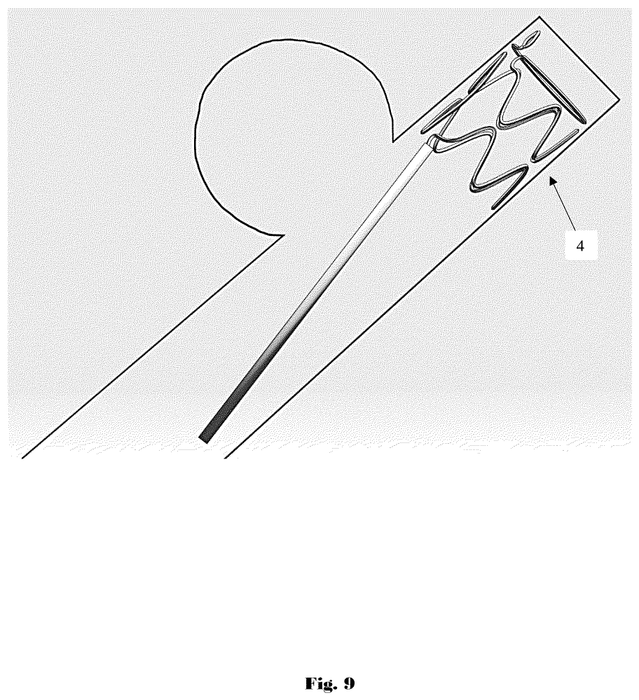

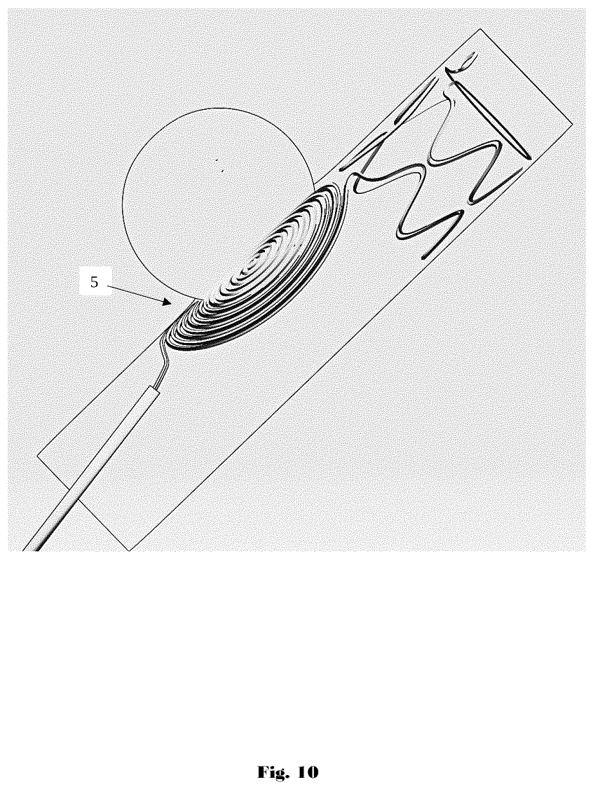

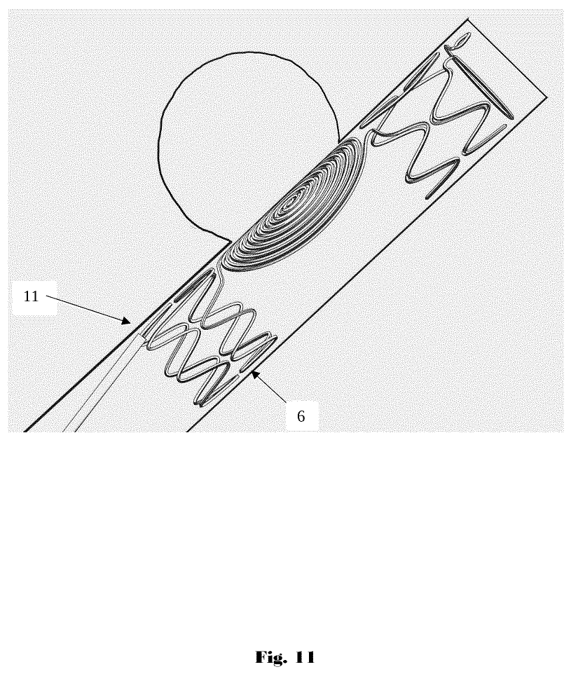

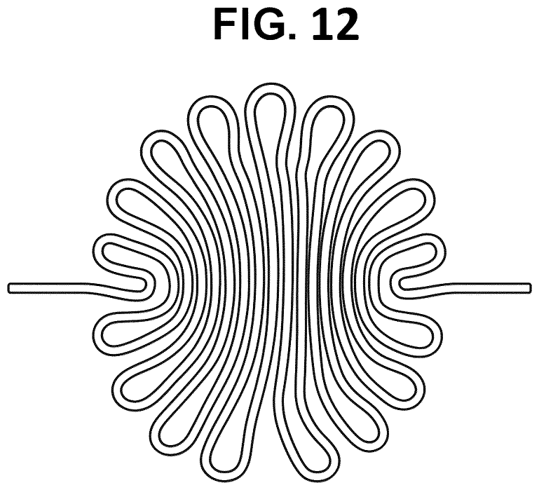

The device is conveyed and maneuvered as a singular filament within a delivery catheter 7, maintaining a rectified configuration that aligns with its predetermined shape-memory design, allowing it to assume the distinct conformations of each of the four components described above, in a sequential manner from distal to proximal upon deployment. See FIG. 8, FIG. 9, FIG. 10 and FIG. 11.

Finally, the device is released. At the most proximal end of the proximal crown, two essential components converge, separated by a bar of a thermal melting polymer: filament and pusher wire junction; these three structures conform the detachment mechanism. The micro guidewire pusher facilitates precise navigation. This detachment mechanism, well-known to seasoned endovascular practitioners, can be either mechanical or thermoelectric. The thermo-electric detachment method is the preferred technique. This method leverages the well-known property of certain polymers that can dissolve when exposed to a high-frequency electric current. This property enables precise and controlled release of endovascular devices, such as stents and occlusion coils, ensuring they are correctly positioned within the body. If the device has not been fully released, it remains fully retrievable, and the operator can retrieve it following their intent.

The device serves as an occluding endovascular system, based on the diverter effect of a spiral barrier 5, which achieves its objective from the vascular lumen and in front of the lesion. The operator does not need to enter the interior of the lesion, as the entire process occurs from within the lumen of the diseased vessel.

The device is manufactured from a single filament, The manufacturing process for this device relies on the remarkable properties of nitinol-a metal alloy composed of nickel and titanium, which is considered the preferred material for the manufacturing process of this device. Nitinol combines nickel (Ni) and titanium (Ti) in specific proportions, and this alloy exhibits a unique property called shape memory effect: when deformed (e.g., compressed or bent), it can return to its original shape upon heating. This behavior is crucial for the functionality of implantable devices. Nitinol also demonstrates superelastic behavior: It can undergo significant deformation without permanent damage. Upon unloading, it reverts to its original shape, and this property is ideal for medical devices, like the presented here, that need flexibility and resilience. Nitinol is preferred for manufacturing various implantable devices. Examples include stents, guidewires, and occlusion devices (like the one described here). Its biocompatibility and mechanical properties make it suitable for use within the human body. In the intricate world of medical materials, nitinol plays a vital role in enhancing patient care.

One or more of the following materials may also be used: stainless steel, cobalt-chromium alloy, titanium or a titanium alloy, tantalum or a tantalum alloy, polymer-based resin or other polymer; it can also be manufactured from various combinations of these alloys. It is well known to those with experience and skill in the art that shape-memory alloys are a special class, as they have a property that makes them superelastic and allows them to “remember” a preselected shape during their manufacturing process, once they are made and deployed from within the catheter that transported them to their destination. Different polymers could also be used in some of the embodiments of this invention, including biodegradable polymers, such as those described below, for purposes of example, but not limited thereto: copolymers of polyglycolic acid or polylactic acid, poly D-lactide, poly D-L lactide, or copolymers obtained from combinations thereof. The occluding mehses (4), which adheres to the metallic structural framework (5), can be made of a material that provides the properties sought in the present invention, and can be selected from a group consisting of: a mesh; a network; a meshed; a membrane; a textile layer; a layer of a shape-memory material; a substantially flat patch of a compressible material; a component obtained from collagen extracellular matrix. As examples of materials for its manufacture, the following list is presented (although the specific material may not be limited to it): a metal alloy, a biocompatible textile material, such as a polymer, among them, but not limited to this list: polycarbonate polyurethanes, polyester polyurethanes, polyether polyurethanes, polysiloxone polyurethanes, or polyurethanes combined with soft elements, polyvinyl alcohols, polystyrene, nylon, polytetrafluoroethylene (PTFE), dacron, polyvinyl acetate, cellulose acetate, ethylene-propylene-diene monomer (EPDM), latex, silicone, polytetrafluoroethylene (PFTE), polyvinyl chloride, polyethylene, polyethylene ethafoam, polyethylene zotefoam. It may also be made from a bioabsorbable polymer, such as copolymers of polyglycolic acid or polylactic acid, poly D-lactide, poly D-L lactide, or copolymers obtained from their combinations, or similar. It can also be formed from the combination of two or more polymers, or from a polymer and a non-polymer. Another material that can be used is an elastomeric polymer, such as expanded polytetrafluoroethylene.

The design of this device, with its controllable porosity, demonstrates a thoughtful approach to addressing vascular lesions. Let's highlight the key points:
The device aims to occlude or isolate defects in the vascular wall (e.g., aneurysm necks), achieving this by strategically positioning the barrier 5, in such a way that allow blood flow to escape from the focal lesion (aneurysm), while they reduce blood flow into the lesion after deployment. This controllable porosity is advantageous over uniform porosity seen in other devices. In this sense, this invention aims to harmonize form and function.
Due to this same low porosity in the area directly related to the focal lesion in the arterial wall, such as the neck of an aneurysm 10, the barrier does not allow gaps or channels through which blood flow enters the interior of the lesion with high velocity, which occurs with other prior art methods (such as coils and the stent-coil combination), and this is the most recognized explanation for the recanalization of the defect in the vascular wall, one of the problems presented with the prior art, which can be significantly reduced or completely avoided by means of the device and method presented in this invention. The micro-slits of the loops that conforms the barrier, once the filament has resumed its original spiral shape, enable it to effectively divert blood flow and promote the healing of the aneurysm. The filament may be of uniform or non-uniform thickness, ranging from 0.005 inches (0.127 mm) to 0.050 inches (1.27 mm); preferably the thickness of the filament will range from 0.015 inches (0.381 mm) to 0.030 inches (0.762 mm). It will generally have a substantial degree of flexibility, elasticity, or resiliency that allows the device to travel in a straight path and to be reshaped once it is released from the catheter that transports it to the blood vessel, the entire procedure taking place within the lumen of the blood vessel, without the need to enter the focal lesion, and without causing damage to the vascular wall. The guide ring, distal and proximal stabilizing crowns, as well as the spiral barrier are substantially straight before their release, and, once the microcatheter is retracted, they acquire their memory-shape to occupy a plane parallel to the axial longitudinal axis of the parent vessel, that is, the vessel that originates the aneurysm.

Incorporating radiopaque markings into the device allows for effective monitoring during transport, deployment, and release. These markers enhance visibility using X-rays. Additionally, coating or plating the device with radiopaque substances (such as tungsten, tantalum, gold, or platinum) ensures clear visualization. The integration of radiopaque dot markers using various methods further enhances its functionality.

The device presented here is manufactured from a filament. In the manufacturing process, said filament must be given the shape of a guide ring 3 at the most distal segment, and sequentially from distal to proximal: distal crowns 4, a spiral barrier 5 and a proximal crowns 6. The crowns formed by the filament, whether straight or given specific shapes, play a crucial role in achieving the desired stability. The distal guide ring, which is the first to be deployed, has a circular shape, and after its deployment, the operator must rotate the filament until the designated mark on the distal end of the guide ring is precisely aligned with the central axis of the lesion's neck, ensuring accurate positioning, since this component of the guide ring is in turn aligned with the center of the spiral barrier. Subsequently, the carrying catheter must be gently retracted to progressively release the filament, allowing it to assume its shape memory and form the remaining three components: distal crowns, spiral barrier and proximal crowns. All the loops of this spiral barrier are formed in the same plane, and therefore the distal spiral ends up being an essentially flat structure, that adapts to the shape of the blood vessel, slightly exceeding the perimeter of the neck of the aneurysm.

At the most proximal end 2 of the proximal stabilizing crowns 3, the device is attached to a soluble polymer rod. This rod is dissolved by a high-frequency electric current transmitted through the pusher wire. The detachment mechanism, familiar to experienced endovascular practitioners, can be either mechanical or thermoelectric.

In relation to the method of use of the device, everything can be condensed into a widely used concept in the endovascular art, such as the deployment of a filament navigated within a microcatheter. This filament, when stripped by the retraction of the microcatheter that contains it, recovers the memory shape with which it was manufactured, to reconstitute itself as the four described components of the device.

The operator begins by navigating a microcatheter, which is positioned at least 4 mm distal to the aneurysm neck, as illustrated in FIG. 8. This strategic positioning ensures optimal coverage and effective deployment of the occlusive barrier, forming a protective shield, as their central purpose is to divert blood flow into the aneurysm, reducing the risk of rupture. To achieve optimal positioning, the operator must rotate the guide ring 3 while visualizing the procedure under high-resolution fluoroscopy, as shown in FIG. 8. When the operator confirms satisfactory alignment of the radiopaque marker 8 on the guide ring 3 with the center of the aneurysm neck, and this design ensures the correct positioning of the spiral barrier, as the radiopaque marker on the guide ring 8 is aligned with the center of the spiral barrier. Once this step is completed, the operator proceeds to unfold the remaining filament that makes up the other components of the device. As the microcatheter is retracted, the filament gradually assumes its memory shape. Initially, it forms the distal stabilizing crowns. Subsequently, the occluding spiral's most peripheral loop emerge, culminating in the formation of the whole spiral 5. As the spiral forms an infinite loop, the central loop returns and expands into progressively larger loops, ultimately covering the entire neck of the aneurysm. The proximal end of this spiral seamlessly continues into the most distal end of the proximal stabilizing crowns. Upon radiological confirmation that all four components have been successfully deployed and the occlusive spiral barrier has been accurately positioned, encompassing the entirety of the aneurysm neck, the operator can proceed with confident with the filament detachment.

It's worth noting that the best available evidence supports using a single endoluminal device for most indications in the context of aneurysms.

Observing the device closely, the operator notes its precise configuration:
The spiral barrier 5 conforms to the edges of the aneurysm neck 10, extending slightly beyond its boundaries for secure positioning.

The stabilizing crowns 3, 6, positioned both distally and proximally, securely engage with the walls of the parent vessel surrounding the aneurysm. Their diameter intentionally exceeds that of the vessel, ensuring a snug fit.

The radiopaque marker 8 of the guide ring 3, at its distal end, is precisely aligned with the center of the aneurysm neck 10. This radiopaque marker 8 of the guide ring 3 is perfectly aligned with the center of the spiral barrier in both the axial and coronal planes. Consequently, aligning it with the center of the aneurysm neck in these planes ensures proper alignment and positioning of the spiral barrier with the aneurysm neck. The guide ring does not come into direct contact with the walls of the parent vessel, as its diameter is slightly undersized.

This meticulous set ensures the successful deployment of the flow-diverting mechanism.

In relation to the dimensions of each of the components of the device, the specifications are crucial for achieving optimal outcomes. Here's how the spiral barrier, strategically deployed to envelop the entire aneurysm neck, should have a diameter approximately between 101 and 120% of the maximum neck diameter. This ensures it rests securely against the intact vascular wall edges from which the aneurysm originates. The distal and proximal stabilizing crowns should be sized at approximately between 105 and 120% of the vessel's lumen diameter where they will be deployed. This, as well as its radial force, ensures optimal adherence to the walls of the parent vessel. The guide ring's inner diameter should be between 0.8 and 0.99 times the diameter of the parent vessel, measured at a point distal to the aneurysm, and avoids contact with the vascular walls to facilitate precise placement and minimize potential endothelial damage and thrombotic complications, when rotating the ring. This, and smooth movements minimizes friction and ensures adequate positioning of the device.

Regarding the effects of the device as a flow diverter restricted to the aneurysm neck, it should be highlighted that the efficacy in altering aneurysmal hemodynamics is predicated on achieving precise spiral dimensions and accurate placement within the aneurysm neck. This enables optimal flow reduction, induces thrombosis, and mitigates the risk of aneurysm progression or rupture, ultimately determining the fate of the aneurysm. This altered flow pattern is beneficial because it decreases the impact force on the aneurysm wall. The spiral flow diverter's presence alters the aneurysm's hemodynamic environment, leading to reduced flow velocities, decreased wall shear stresses, and increased intra-aneurysmal residence times. These changes can induce aneurysmal involution, marked by gradual shrinkage or collapse. Additionally, the decreased inflow jet velocity promotes thrombosis, leading to stable clot formation within the aneurysm sac, which in turn facilitates aneurysmal stabilization, healing, and reduced rupture risk.

Over subsequent weeks or months, the aneurysm's response to the altered flow conditions becomes evident. The combination of reduced inflow forces and potential thrombosis enhances the chances of long-term success. Regular follow-up imaging helps monitor the aneurysm's evolution and assess its stability.

The pusher wire allows the device to be navigated endovascularly inside the microcatheter to access the parent vessel, focus on the segment immediately distal to the neck of the aneurysm. The guidewire is manufactured as a metal wire with a diameter between 0.014 and 0.016 inches (0.36 and 0.041 mm) and a length between 160 and 200 centimeters. Its proximal end is in the hands of the operator; the guidewire contacts the proximal end of the filament. Once the union guidewire-filament is disintegrated by any of the mechanisms for detachment of endovascular implants known in the art, whether hydraulic, thermal, electrothermal, mechanical or electrolytic, the pusher guidewire is released from its contact with the device One or more connectors may be used to attach the device to the navigation or supply pusher wire in such a way that its release can be caused in a controlled manner.

The type of catheter used in the endovascular transport and navigation for the present invention can have any dimension that allows it to reach through the body vasculature to the aneurysmal lesion and reach its final position, with the following range of dimensions of its external diameter: from 1.5 French (0.019 inches-0.5 mm), up to 3 French (0.039 inches-1 mm). Most often a 2 French catheter (0.026 inch-0.67 mm) will be used.

The method by which the device described in the present invention achieves the effect of occlusion or isolation from the circulation of an aneurysm, is explained below:

The operator employs a set of purpose-designed elements to deploy and stabilize a spiral barrier 5 within the neck region 10 of an aneurysm. These elements function as an occlusive mechanism, effectively addressing the aneurysm.
The device includes four components, enunciated from distal to proximal: the guide ring 3, the distal stabilizing crowns 4, the spiral barrier 5 and the proximal stabilizing crowns 6. The device boasts a unified architecture, wherein all constituent parts are derived from a single filament. This filament undergoes a shape memory process, whereby discrete sections are programmed with specific shape memory alloys (SMAs) or polymers, granting each region unique properties. This cutting-edge design facilitates the seamless fusion of multiple functional elements, ensuring a cohesive and robust device.
The entire device is packed inside a microcatheter 7, 11 using endovascular techniques. The microcatheter 7 is advanced until it is positioned in the distal segment of the aneurysm neck 10.
Deployment Sequence: Under fluoroscopy guidance, the operator advances the microcatheter distally to the aneurysm's neck. Initially, the carrier microcatheter is slowly and carefully retracted, exposing only the guide ring component, as illustrated in FIG. 8. At this stage, rotating the device via the guidewire allows for precise alignment of the tiny circular shape 8 at the distal end 1 of the guide ring 3 with the center of the aneurysm neck 10. Once the distal ring is properly positioned, the operator must slowly and gently retract the microcatheter. This action will sequentially deploy the remaining components of the device, ensuring their correct placement and functionality: first the distal stabilizing crowns 4, as shown in FIG. 9, followed by the occluding spiral barrier 5, as illustrated in FIG. 10, and finally the proximal stabilizing crowns 6, as shown in FIG. 11. The spiral barrier 5, acting as the device's occluding component, effectively covers the entire neck area of the aneurysm, as shown in FIG. 11.
Release and targeted coverage: the pusher wire detachment system 11 is activated, releasing the device. The device remains within the patient's vascular segment, covering the predefined area of the aneurysm's neck. Importantly, the operator achieves this without directly manipulating the lesion itself or affecting the aneurysm wall. This meticulous procedure allows the doctor who acts as an operator to precisely position the device, cover the targeted stent area, and achieve effective flow diversion without disturbing either the aneurysm or the parent vessel.
   Principio Del formularioFinal Del Formulario

What is claimed is:

1. A device for repairing aneurysms, the device comprising:
a. a filamentous material adapted for insertion into the parent vessel of an aneurysm,
b. said filamentous material made of a biocompatible material with shape memory,
c. said filamentous material adopting, once deployed, the shape of a stent-like device with a spiral barrier in the middle third of its length and a guide ring at its distal end,
d. means for intraoperative endoluminal positioning of said filamentous material to said parent vessel, comprising elements configured to securely affix the spiral barrier to the neck borders of the aneurysm,
e. the first deployed element of said filamentous material being the distal component of said device, wherein said distal component comprises a partially closed ring with a small ring at its first free end, oriented towards the distal segment of said vessel, and with its second end directed towards the proximal segment of said parent vessel, seamlessly integrating with the second deployed element of said device,
f. said second deployed element of said filamentous material being the continuation of the proximal end of said first element, configuring the second component of the device, which consists of one or a plurality of stent-like crowns, each forming a partially closed annulus, said filamentous material within each crown adopting a sinusoidal configuration and transitioning seamlessly at its proximal end into the third deployed element, creating a continuous, unbroken structure,
g. said third deployed element being the continuation of the proximal end of said second component, conforming the third component of said device, exhibiting an Archimedean spiral geometry, characterized by a continuously decreasing radius that converges towards the central axis, reaching a minimum loop diameter before reversing direction and expanding outward in progressively increasing spiral turns, ultimately attaining a diameter equivalent to the first loop, said spiral terminating at its proximal end, which forms a seamless junction with the fourth deployed element,
h. said fourth deployed element being the continuation of the proximal end of said third component, conforming the fourth component of said device, consisting of one or a plurality of stent-like crowns, each forming a partially closed annulus, said filamentous material within each crown adopting a sinusoidal configuration, said fourth component having at its proximal end a design to being the interface with the device's detachment and release system.

2. The device for repairing aneurysms of claim 1, wherein the diameter of said first component corresponds to 80 to 99% of the diameter of the parent vessel immediately distal to the aneurysm neck.

3. The device for repairing aneurysms of claim 1, wherein the diameter of said crowns of said second and fourth components corresponds to 101% to 120% of the mean diameter of said parent vessel along the segment including said aneurysm neck.

4. The device for repairing aneurysms of claim 1, wherein the diameter of the outermost loops of said third component corresponds to 101% to 120% of the mean diameter of the aneurysm neck area.

5. The device for repairing aneurysms of claim 1, wherein said third component, referred as said spiral barrier, exerts a flow diversion effect by occupying between 60 and 100% of the area of said aneurysm neck area.

6. The device for repairing aneurysms of claim 1, wherein the first component, referred to as the guide ring, features a small circular point with a highly visible radiopaque marker.

7. The device for repairing aneurysms of claim 1, wherein said circular point of said guide ring is aligned with the central point of said spiral barrier, with “central” referring to the axial, coronal, and sagittal planes.

8. The device for repairing aneurysms of claim 1, wherein said third component, referred as said spiral barrier, conforms a flow diverter obstacle in the aneurysm neck area.

9. A method of placing a device for repairing aneurysms, the method comprising the steps of:
a. providing a filamentous material adapted for insertion into the parent vessel of an aneurysm,
b. providing said filamentous material made of a biocompatible material with shape memory,
c. providing said filamentous material adopting, once deployed, the shape of a stent-like device with a spiral barrier in the middle third of its length and a guide ring at its distal end,
d. providing means for intraoperative endoluminal positioning of said filamentous material to said parent vessel, comprising elements configured to securely attach said spiral barrier to the neck of said aneurysm,
e. providing the first deployed element of said filamentous material being the distal component of said device, wherein said distal component comprises a partially closed ring with a small ring at its first free end, oriented towards the distal segment of said vessel, and with its second end directed towards the proximal segment of said parent vessel, seamlessly integrating with the second deployed element of said device,
f. providing said second deployed element of said filamentous material being the continuation of the proximal end of said first element, configuring the second component of the device, which consists of one or a plurality of stent-like crowns, each forming a partially closed annulus, said filamentous material within each crown adopting a sinusoidal configuration and transitioning seamlessly at its proximal end into the third deployed element, creating a continuous, unbroken structure,
g. providing said third deployed element being the continuation of the proximal end of said second component, conforming the third component of said device, exhibiting an Archimedean spiral geometry, characterized by a continuously decreasing radius that converges towards the central axis, reaching a minimum loop diameter before reversing direction and expanding outward in progressively increasing spiral turns, ultimately attaining a diameter equivalent to the first loop, said spiral terminating at its proximal end, which forms a seamless junction with the fourth deployed element,
h. providing said fourth deployed element being the continuation of the proximal end of said third component, conforming the fourth component of said device, consisting of one or a plurality of stent-like crowns, each forming a partially closed annulus, said filamentous material within each crown adopting a sinusoidal configuration, said fourth component having at its proximal end a design to being the interface with the device's detachment and release system.

10. The method according to claim 9, wherein the diameter of said first component corresponds to 80 to 99% of the diameter of the parent vessel immediately distal to the aneurysm neck.

11. The method according to claim 9, wherein the diameter of said crowns of said second and fourth components corresponds to 101% to 120% of the mean diameter of said parent vessel along the segment including said aneurysm neck.

12. The method according to claim 9, wherein the diameter of the outermost loops of said third component corresponds to 101% to 120% of the mean diameter of the aneurysm neck area.

13. The method according to claim 9, wherein said third component, referred as said spiral barrier, exerts a flow diversion effect by occupying between 60 and 100% of the area of said aneurysm neck area.

14. The method according to claim 9, wherein the first component, referred to as the guide ring, features a small circular point with a highly visible radiopaque marker.

15. The method according to claim 9, wherein said circular point of said guide ring is aligned with the central point of said spiral barrier, with “central” referring to the axial, coronal, and sagittal planes.

16. The method according to claim 9, wherein said circular point of said guide ring is aligned by the operator with the central point of said aneurysm neck, with “central” referring to the axial, coronal, and sagittal planes.

17. The method according to claim 9, wherein said third component, referred as said spiral barrier, conforms a flow diverter obstacle in the aneurysm neck area.

18. The device for repairing aneurysms of claim 1, wherein said third component may alternatively adopt a petal-shaped sinusoidal geometry, comprising a plurality of lobes that gradually increase in amplitude toward a central region and then decrease symmetrically, forming a petal-like occluding pattern.

19. The method according to claim 9, wherein said third component may alternatively adopt a petal-shaped sinusoidal geometry, comprising a plurality of lobes that gradually increase in amplitude toward a central region and then decrease symmetrically, forming a petal-like occluding pattern.
